(19) United States
(12) Patent Application Publication
(10) Pub. No.: US 2012/0296131 A1
(43) Pub. Date: Nov. 22, 2012

(54) METHOD FOR ALKYLATION OF TOLUENE IN A PRE-EXISTING DEHYDROGENATION PLANT

(75) Inventors: James R. Butler, Spicewood, TX (US); Sivadinarayana Chinta, Missouri City, TX (US); Joseph E. Pelati, Houston, TX (US)

(73) Assignee: FINA TECHNOLOGY, INC., Houston, TX (US)

(21) Appl. No.: 13/457,507

(22) Filed: Apr. 27, 2012

Related U.S. Application Data
(60) Provisional application No. 61/488,779, filed on May 22, 2011.

(51) Int. Cl. C07C 2/88

(52) U.S. Cl. 585/323; 585/437; 585/403

(57) ABSTRACT

A process for making styrene in a pre-existing facility including an infrastructure capable of producing styrene, wherein the infrastructure includes at least one dehydrogenation unit. The process includes coupling an alkylation unit including an alkylation reactor to the infrastructure and contacting toluene with a C₁ source in the presence of a first catalyst and a co-feed in the alkylation reactor to form a first product stream comprising styrene and ethylbenzene. The styrene and ethylbenzene from the first product stream are routed for further processing to a portion of the pre-existing facility.

### CROSS-REFERENCE TO RELATED APPLICATIONS

This application claims priority to U.S. Provisional Patent No. 61/488,779 filed on May 22, 2011.

### FIELD

The present invention relates to a method for the production of styrene and ethylbenzene. More specifically, the invention relates to the alkylation of toluene with methanol and/or formaldehyde in a pre-existing dehydrogenation plant to produce styrene and ethylbenzene.

### BACKGROUND

Styrene is a monomer used in the manufacture of many plastics. Styrene is commonly produced by making ethylbenzene, which is then dehydrogenated to produce styrene. Ethylbenzene is typically formed by one or more aromatic conversion processes involving the alkylation of benzene.

Aromatic conversion processes, which are typically carried out utilizing a molecular sieve type catalyst, are well known in the chemical processing industry. Such aromatic conversion processes include the alkylation of aromatic compounds such as benzene with ethylene to produce alkyl aromatics such as ethylbenzene. Typically an alkylation reactor, which can produce a mixture of monoalkyl and polyalkyl benzenes, will be coupled with a transalkylation reactor for the conversion of polyalkyl benzenes to monoalkyl benzenes. The transalkylation process is operated under conditions to cause disproportionation of the polyalkylated aromatic fraction, which can produce a product having an enhanced ethylbenzene content and reduced polyalkylated content. When both alkylation and transalkylation processes are used, two separate reactors, each with its own catalyst, can be employed for each of the processes.

Ethylene is obtained predominantly from the thermal cracking of hydrocarbons, such as ethane, propane, butane, or naphtha. Ethylene can also be produced and recovered from various refinery processes. Thermal cracking and separation technologies for the production of relatively pure ethylene can account for a significant portion of the total ethylbenzene production costs.

Benzene can be obtained from the hydrodealkylation of toluene that involves heating a mixture of toluene with excess hydrogen to elevated temperatures (for example 500° C. to 600° C.) in the presence of a catalyst. Under these conditions, toluene can undergo dealkylation according to the chemical equation: C₆H₅CH₃+H₂→C₆H₆+CH₄. This reaction requires energy input and as can be seen from the above equation, produces methane as a byproduct, which is typically separated and may be used as heating fuel for the process.

In view of the above, it would be desirable to have a process of producing styrene and/or ethylbenzene that does not rely on thermal crackers and expensive separation technologies as a source of ethylene. It would also be desirable if the process was not dependent upon ethylene from refinery streams that contain impurities which can lower the effectiveness and can contaminate the alkylation catalyst. It would further be desirable to avoid the process of converting toluene to benzene with its inherent expense and loss of a carbon atom to form methane. It would be desirable to produce styrene without the use of benzene and ethylene as feedstreams. It would also be desirable to produce styrene and/or ethylbenzene in one reactor without the need for separate reactors requiring additional separation steps. Furthermore, it is desirable to achieve a process having a high yield and selectivity to styrene and ethylbenzene. Even further, it is desirable to achieve a process having a high yield and selectivity to styrene such that the step of dehydrogenation of ethylbenzene to produce styrene can be reduced. It is further desirable to be able to produce a catalyst having the properties desired without involving flammable materials and/or intermediate drying steps.

### SUMMARY

The present invention in its many embodiments relates to a process of making styrene.

In an embodiment, either by itself or in combination with any other embodiment, a process is provided for making styrene in a pre-existing facility including an infrastructure capable of producing styrene. The infrastructure includes at least one dehydrogenation unit and the process includes coupling an alkylation unit including an alkylation reactor to the infrastructure and contacting toluene with a C₁ source in the presence of a first catalyst and a co-feed in the alkylation reactor to form a first product stream including styrene and ethylbenzene. The styrene and ethylbenzene from the first product stream are routed for further processing to a portion of the pre-existing facility. The C₁ source can be selected from the group consisting of methanol, formaldehyde, formalin, trioxane, methylformcel, paraformaldehyde, methylal, dimethyl ether, and combinations thereof.

In an embodiment, either by itself or in combination with any other embodiment, the process further includes forming the co-feed from the separation of a mixture of hydrogen and carbon monoxide. The mixture is separated from an initial product stream including styrene and ethylbenzene, and the initial product stream is formed from toluene contacting the C₁ source in the presence of an initial catalyst in the alkylation reactor. The co-feed can include carbon monoxide.

In an embodiment, either by itself or in combination with any other embodiment, the infrastructure includes a plurality of infrastructure reactors connected in parallel with the alkylation reactor. Optionally, the infrastructure reactors are configured to process at least an ethylene feedstock. The alkylation unit can include a ceramic membrane configured to separate the mixture of hydrogen and carbon monoxide.

In an embodiment, either by itself or in combination with any other embodiment, the first catalyst includes at least one promoter on a support material. The promoter can be selected from the group consisting of Co, Mn, Ti, Zr, V, Nb, K, Cs, Ga, B, P, Rb, Ag, Na, Cu, Mg, Fe, Mo, Ce, and combinations thereof. Optionally, the promoter is selected from the group consisting of Ce, Cu, P, Cs, B, Co, Ga, and combinations thereof. The support material can include a zeolite. Optionally, the first catalyst includes B and Cs supported on a zeolite.

Another embodiment of the present invention includes a process for making ethylbenzene and/or styrene including reacting toluene and methanol in the presence of a co-feed in one or more reactors to form a first product stream including one or more of ethylbenzene, styrene, toluene, methanol, hydrogen, and carbon monoxide; separating the ethylbenzene from the first product stream; sending the ethylbenzene to a portion of a pre-existing facility; and forming styrene from the ethylbenzene in the pre-existing facility.

In an embodiment, either by itself or in combination with any other embodiment, the process further includes separating a mixture of the hydrogen and carbon monoxide from the first product stream; separating at least a portion of the carbon monoxide from the mixture and recycling the carbon monoxide as the co-feed; and sending ethylbenzene to a dehydrogenation unit in the pre-existing facility. Styrene is formed from the dehydrogenation of ethylbenzene in the dehydrogenation unit. Optionally, the process includes separating styrene from the first product stream and sending the styrene and ethylbenzene to a separation unit in the pre-existing facility.

In yet another embodiment of the present invention, a method of revamping an existing styrene production facility includes coupling one or more reactors to an existing styrene production facility. The reactor is capable of reacting toluene with methanol in the presence of a co-feed to produce a first product stream including ethylbenzene and/or styrene. The method can further include sending the first product stream including ethylbenzene and/or styrene to the existing styrene production facility for further processing to form styrene.

In an embodiment, either by itself or in combination with any other embodiment, the existing styrene production facility includes a separation apparatus to remove at least a portion of any benzene from the first product stream, an alkylation reactor to form ethylbenzene by reacting benzene and polyethylbenzene, and a dehydrogenation reactor to form styrene by dehydrogenating ethylbenzene. Optionally, the first product stream includes one or more of benzene, toluene or methanol and at least a portion of the methanol is separated from the first product stream and recycled to the one or more reactors. Optionally, the first product stream includes one or more of hydrogen, carbon monoxide, toluene or methanol and at least a portion of the carbon monoxide is separated from the first product stream and recycled to the one or more reactors.

The various aspects of the present invention can be joined in combination with other aspects of the invention and the listed embodiments herein are not meant to limit the invention. All combinations of aspects of the invention are enabled, even if not given in a particular example herein.

### BRIEF DESCRIPTION OF DRAWINGS

FIG. 1 is a schematic block diagram illustrating a conventional process for making styrene and ethylbenzene.

FIG. 2 is a schematic block diagram illustrating a process for making styrene and ethylbenzene according to an embodiment of the present invention.

FIG. 3 is a schematic block diagram illustrating a process for making styrene and ethylbenzene according to an embodiment of the present invention.

### DETAILED DESCRIPTION

In a non-limiting embodiment, either by itself or in combination with any other aspect of the invention, toluene is reacted with a carbon source capable of coupling with toluene to form ethylbenzene or styrene, which can be referred to as a C₁ source, in the presence of a co-feed to produce styrene and ethylbenzene. In an embodiment, the C₁ source includes methanol or formaldehyde or a mixture of the two. In an alternative embodiment, toluene is reacted with one or more of the following: formalin, trioxane, methylformcel, paraformaldehyde and methylal. In a further embodiment, the C₁ source is selected from the group consisting of methanol, formaldehyde, formalin (37-50% H₂CO in solution of water and methanol), trioxane (1,3,5-trioxane), methylformcel (55% H₂CO in methanol), paraformaldehyde and methylal (dimethoxymethane), dimethyl ether, and combinations thereof.

Formaldehyde can be produced either by the oxidation or dehydrogenation of methanol. In an embodiment, formaldehyde is produced by the dehydrogenation of methanol to produce formaldehyde and hydrogen gas. This reaction step produces a dry formaldehyde stream that may be preferred, as it would not require the separation of the water prior to the reaction of the formaldehyde with toluene. The dehydrogenation process is described in the equation below:

CH₃OH→CH₂O+H₂

Formaldehyde can also be produced by the oxidation of methanol to produce formaldehyde and water. The oxidation of methanol is described in the equation below:

2CH₃OH+O₂→2CH₂O+2H₂O

In the case of using a separate process to obtain formaldehyde, a separation unit may then be used in order to separate the formaldehyde from the hydrogen gas or water from the formaldehyde and unreacted methanol prior to reacting the formaldehyde with toluene for the production of styrene. This separation would inhibit the hydrogenation of the formaldehyde back to methanol. Purified formaldehyde could then be sent to a styrene reactor. Although the reaction has a 1:1 molar ratio of toluene and the C₁ source, the ratio of the C₁ source and toluene feedstreams is not limited within the present invention and can vary depending on operating conditions and the efficiency of the reaction system. If excess toluene or C₁ source is fed to the reaction zone, the unreacted portion can be subsequently separated and recycled back into the process. In one embodiment the ratio of toluene:C₁ source can range from between 100:1 to 1:100. In alternate embodiments the ratio of toluene:C₁ source can range from 50:1 to 1:50; from 20:1 to 1:20; from 10:1 to 1:10; from 5:1 to 1:5; from 2:1 to 1:2. In a specific aspect, the ratio of toluene:C₁ source can range from 2:1 to 5:1.

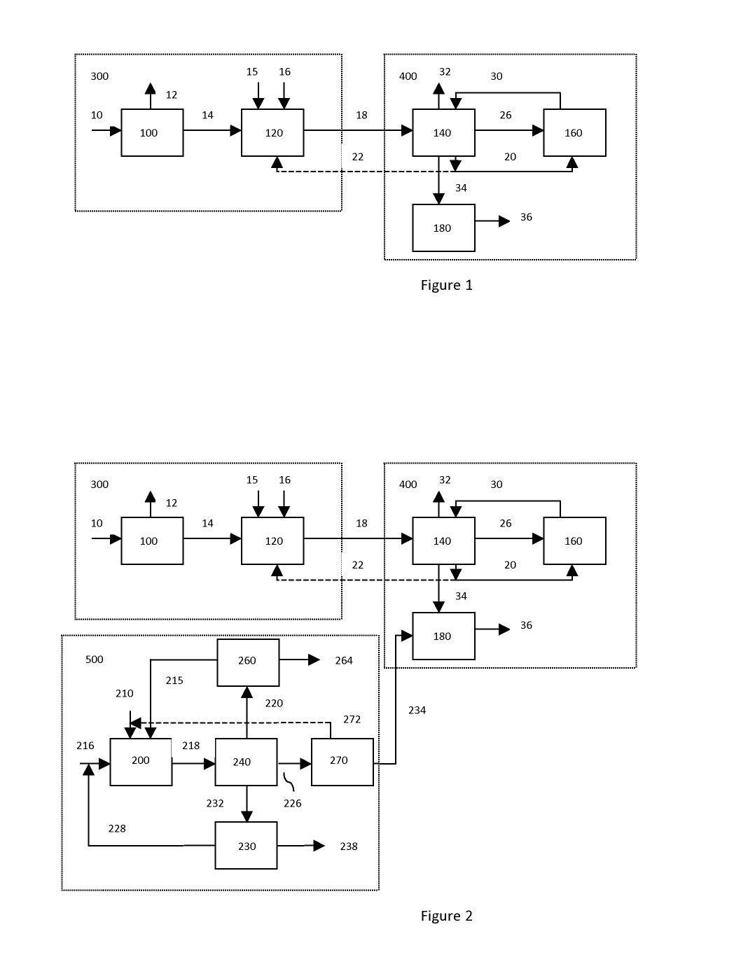

Turning now to the drawings and referring first to FIG. 1, there is illustrated a schematic block diagram of a conventional alkylation/transalkylation process. The process may be carried out utilizing at least a portion of an infrastructure in an existing facility. A feed stream of toluene is supplied via line (10) to reactive zone (100) which produces product streams of methane via line (12) and benzene via line (14). The benzene via line (14) along with ethylene via line (16) and optional co-feeds or additives via line (15) is supplied to an alkylation reactive zone (120), which produces ethylbenzene and other products which are sent via line (18) to a separation zone (140). The separation zone (140) can remove benzene via line (20) and send it to a transalkylation reaction zone (160). The benzene can also be partially recycled via line (22) to the alkylation reactive zone (120). The separation zone (140) can also remove polyethylbenzenes via line (26) which are sent to the transalkylation reaction zone (160) to produce a product with increased ethylbenzene content that can be sent via line (30) to the separation zone (140). Other byproducts can be removed from the separation zone (140) as shown by line (32). These byproducts can include methane and other hydrocarbons that can be recycled within the process, used as fuel gas, flared, or otherwise disposed of Ethylbenzene can be removed from the separation zone (140) via line (34) and sent to a dehydrogenation zone (180) to produce styrene product that can be removed via line (36).

The front end of the process (300), designated by the dashed line, includes the initial toluene to benzene reactive zone (100) and the alkylation reactive zone (120). It can be seen that the input streams to the front end (300) can include toluene via line (10) and ethylene via line (16). There can also be input streams of benzene from alternate sources other than from a toluene reaction, shown as reactive zone (100), although they are not shown in this figure. The output streams include the methane via line (12) which is produced during the conversion of toluene to benzene in reactive zone (100) and the product stream containing ethylbenzene via line (18) that is sent to the back end of the process (400). The back end (400) includes the separation zone (140), the transalkylation reaction zone (160), and the dehydrogenation zone (180).

Turning now to FIG. 2, there is illustrated a schematic block diagram of one embodiment of the present invention. Feed streams of toluene supplied via line (210) and methanol supplied via line (216) are supplied to a toluene alkylation unit (500) including a reactive zone (200), which produces ethylbenzene along with other products, which can include styrene. In some embodiments, an input stream of carbon monoxide (215) may be supplied to the reactive zone (200). In an alternate embodiment, an input stream of carbon monoxide and hydrogen may be supplied to the reactive zone (200). The output from the reactive zone (200) includes a product containing ethylbenzene and styrene, which is supplied via line (218) to a separation zone (240). The separation zone (240) can separate ethylbenzene, styrene, and unreacted toluene from the product via line (226) which can be sent to separation zone (270).

The separation zone (240) can also separate carbon monoxide and hydrogen that may be present via line (220) which can be sent to a separation zone (260). The separation zone (260) can include a ceramic membrane capable of separating the hydrogen from the carbon monoxide. Optionally, the separation zone can include a Pd alloy membrane capable of separating the hydrogen from the carbon monoxide. The carbon monoxide can be sent via line (215) to the reactive zone (200) as a co-feed. The hydrogen may be sent via line (264) to a flare, used as fuel gas, or otherwise for disposed of in an appropriate manner. Other byproducts can be removed from the separation zone (240) via line (232) and sent to separation zone (230). These byproducts can include methanol and water, wherein methanol may be separated via line (228) and be recycled within the process and fed back to the reactive zone (200). Water may be separated from separation zone (230) via line (238) and sent for further process treatment.

Ethylbenzene can be removed from the separation zone (270) via line (234) and sent to the dehydrogenation zone (180) to produce styrene product that can be removed via line (36). Any styrene that is produced from the reactive zone (200) can be separated in the separation zone (270) and sent to the dehydrogenation zone (180) via line (234) along with the ethylbenzene product stream, or can be separated as its own product stream, (not shown), bypassing the dehydrogenation zone (180) and added to the styrene product in line (36). Unreacted toluene present in the separation zone (270) may be separated via line (272) and fed back into the reactive zone (200).

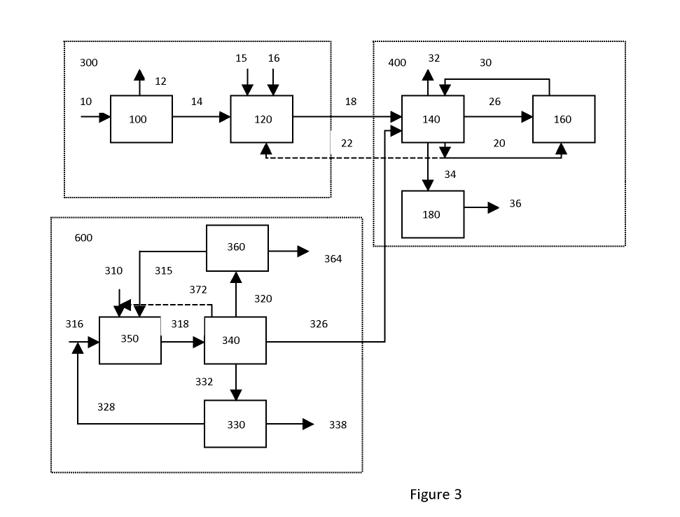

Turning now to the embodiment shown in FIG. 3, feed streams of toluene supplied via line (310) and methanol supplied via line (316) are supplied to a toluene alkylation unit (600) including a reactive zone (350), which produces ethylbenzene along with other products, which can include styrene. In some embodiments, an input stream of carbon monoxide (315) may be supplied to the reactive zone (350). In an alternate embodiment, an input stream of carbon monoxide and hydrogen may be supplied to the reactive zone (350). The output from the reactive zone (350) includes a product containing ethylbenzene and styrene, which is supplied via line (318) to a separation zone (340). The separation zone (340) can separate ethylbenzene and styrene from the product via line (326) which can be sent to separation zone (140). Any unreacted toluene may be separated via line (372) from the separation zone (340) and recycled to the reactive zone (350).

The separation zone (340) can also separate carbon monoxide and hydrogen that may be present via line (320) which can be sent to a separation zone (360). The separation zone (360) can include a ceramic membrane capable of separating the hydrogen from the carbon monoxide. The carbon monoxide can be sent via line (315) to the reactive zone (350) as a co-feed. The hydrogen may be sent via line (364) to a flare, used as fuel gas, or otherwise disposed of in an appropriate manner. Other byproducts can be removed from the separation zone (340) via line (332) and sent to separation zone (330). These byproducts can include methanol and water, wherein methanol may be separated via line (328) and be recycled within the process and fed back to the reactive zone (350). Water may be separated from separation zone (330) via line (338) and sent for further process treatment.

Ethylbenzene can be removed from the separation zone (140) in the back end (400) via line (34) and sent to the dehydrogenation zone (180) to produce styrene product that can be removed via line (36). Any styrene that is produced from the reactive zone (350) can be separated in the separation zone (140) and sent to the dehydrogenation zone (180) via line (34) along with the ethylbenzene product stream, or can be separated as its own product stream, (not shown), bypassing the dehydrogenation zone (180) and added to the styrene product in line (36). As shown in FIG. 3, the embodiment may provide cost savings in that the equipment in the alkylation unit may be reduced with the back end (400) of the pre-existing facility used for the remainder of the styrene production.

The front ends (500, 600) of the processes shown in FIGS. 2 and 3 each include toluene alkylation units respectively including an initial toluene and methanol reactive zone (200, 350). The input streams to the front end (500, 600) are toluene via line (210, 310) and methanol via line (216, 316) and, optionally, carbon monoxide or carbon monoxide and hydrogen, via line (215, 315). The front end (300) of the conventional process can be optional if either front ends (500, 600) of the embodiments of the invention are used.

In the embodiment of FIG. 2, the output stream is further separated, resulting in a product containing ethylbenzene via line (234) that is sent to the back end of the process (400). In FIG. 3, the output stream is further separated, resulting in a product containing ethylbenzene via line (326) that is sent to the back end of the process (400). The back end (400) includes the separation zone (140), the alkylation reaction zone (160), and the dehydrogenation zone (180).

A comparison of the front end (300) of the conventional process shown in FIG. 1 against the front ends (500, 600) of the embodiments of the invention shown in FIG. 2 and FIG. 3 can illustrate some of the features of the present invention. The front ends (500, 600) of the embodiments of the invention shown in FIG. 2 and FIG. 3 have a single reactive zone (200, 350) rather than the two reactive zones, reactive zone (100) and alkylation reactive zone (120), contained within the front end (300) shown in FIG. 1. The reduction of one reactive zone can have a potential cost savings and can simplify the operational considerations of the process.

Each front end (300, 500, 600) has an input stream of toluene, shown as lines (10), (210), and (310), respectively. The conventional process shown in FIG. 1 has an input stream of ethylene (16) and a byproduct stream of methane (12). The embodiments of the invention shown in FIG. 2 and FIG. 3 have an input stream of methanol (216, 316). The feed stream of ethylene (16) is replaced by the feed stream of methanol (216, 316), which is typically a lower value commodity, and should result in a cost savings. Rather than generating methane as a byproduct (12) which would have to be separated, handled and disposed of, the present invention utilizes methanol as a feedstock (216, 316) to the reaction zone (200, 350).

Looking now at the back end (400) of the conventional process shown in the Figures, a further benefit of the present invention is shown. It can be seen that the back end (400) of the conventional process shown in FIG. 1 remains the same in FIGS. 2 and 3, therein maintaining the separation zone (140), the alkylation reaction zone (160), and the dehydrogenation zone (180) of the pre-existing facility, wherein the zones are interconnected in the same or essentially the same manner. This aspect of the present invention can enable the front end of a facility to be modified in a manner consistent with an embodiment of the invention, while the back end remains essentially unchanged. A revamp of an existing ethylbenzene or styrene production facility can be accomplished by installing a new front end or modifying an existing front end in a manner consistent with the invention and delivering the product of the altered front end to the existing back end of the facility to complete the process in essentially the same manner as before. The ability to revamp an existing facility and convert from a toluene/ethylene feedstock to a toluene/methanol feedstock or to add a toluene/methanol feedstock component to the existing facility by the modification of, or addition to, the front end of the facility while retaining the existing back end can have significant economic advantages.

The reactive zones (200, 350) of the present invention each can comprise one or more single or multi-stage reactors. In one embodiment, the reactive zones (200, 350) each can have a plurality of series-connected reactors. Additionally and in the alternative, the reactors in each reactive zone can be arranged in a parallel manner. There can also be embodiments having multiple series-connected reactors that are arranged in a parallel manner. In an embodiment, the reactive zones (200, 350) can be operated at temperature and pressure conditions to enable the reaction of methanol and toluene to form ethylbenzene, and at a feed rate to provide a space velocity enhancing ethylbenzene production while retarding the production of xylene or other undesirable products. The reactants, toluene and methanol in an embodiment, can be added to the plurality of series-connected reactors in a manner to enhance ethylbenzene production while retarding the production of undesirable products. For example toluene and/or methanol can be added to any of the plurality of series-connected reactors as needed to enhance ethylbenzene production.

In an embodiment, the reactive zone (200, 350) is arranged in a parallel manner with the reactive zone (100), such that the reactive zones are configured in a swing manner. Operating in this manner, ethylbenzene may be manufactured in a continuous manner, wherein the reactive zone (100) may be brought online when reactive zone (200, 350) is taken off-line. Such an embodiment may be advantageous also as the reactive zones could be swung depending on the cost of feedstock. In alternate embodiment, the reactive zone (200, 350) is operated simultaneously with the reactive zone (100).

The reactive zones (200, 350) can be operated in the vapor phase. One embodiment can be operated in the vapor phase within a pressure range of 0.1 atm to 1000 psig. Another embodiment can be operated in the vapor phase within a pressure range of 0.1 atm to 500 psig. Another embodiment can be operated in the vapor phase within a pressure range of 0.1 atm to 300 psig. Another embodiment can be operated in the vapor phase within a pressure range of 0.1 atm to 150 psig.

The operating conditions of the reactors and separators will be system specific and can vary depending on the feedstream composition and the composition of the product streams. The alkylation reactor for the reaction of a C₁ source including methanol to formaldehyde and the reaction of toluene with formaldehyde will operate at elevated temperatures and may contain a basic or neutral catalyst system. The temperature can range in a non-limiting example from 250° C. to 750° C., optionally from 300° C. to 500° C., optionally from 375° C. to 450° C. The pressure can range in a non-limiting example from 0.1 atm to 70 atm, optionally from 0.1 atm to 35 atm, optionally from 0.1 atm to 10 atm, optionally from 0.1 atm to 5 atm.

Improvement in side chain alkylation selectivity may be achieved by treating a molecular sieve zeolite catalyst with chemical compounds to inhibit the external acidic sites and minimize aromatic alkylation on the ring positions. Another means of improvement of side chain alkylation selectivity can be to inhibit overly basic sites, such as for example with the addition of a boron compound. Another means of improvement of side chain alkylation selectivity can be to impose restrictions on the catalyst structure to facilitate side chain alkylation. In one embodiment the catalyst used in an embodiment of the present invention is a basic or neutral catalyst.

The catalytic reaction systems suitable for this invention can include one or more of the zeolite or amorphous materials modified for side chain alkylation selectivity. A non-limiting example can be a zeolite promoted with one or more of the following: Co, Mn, Ti, Zr, V, Nb, K, Cs, Ga, B, P, Rb, Ag, Na, Cu, Mg, Fe, Mo, Ce, or combinations thereof. In an embodiment, the zeolite can be promoted with one or more of Ce, Cu, P, Cs, B, Co, or Ga, or combinations thereof. The promoter can exchange with an element within the zeolite or amorphous material and/or be attached to the zeolite or amorphous material in an occluded manner. In an aspect the amount of promoter is determined by the amount needed to yield less than 0.5 mol % of ring alkylated products such as xylenes from a coupling reaction of toluene and a C₁ source.

In an embodiment, the catalyst contains greater than 0.1 wt % of at least one promoter based on the total weight of the catalyst. In another embodiment, the catalyst contains up to 5 wt % of at least one promoter. In a further embodiment, the catalyst contains from 0.1 to 3 wt % of at least one promoter. In an aspect, the at least one promoter is boron.

Zeolite materials suitable for this invention may include silicate-based zeolites and amorphous compounds such as faujasites, mordenites, etc. Silicate-based zeolites are made of alternating SiO₂ and MOₓ tetrahedra, where M is an element selected from the Groups 1 through 16 of the Periodic Table (new IUPAC). These types of zeolites have 4, 6, 8, 10, or 12-membered oxygen ring channels. An example of zeolites of this invention can include faujasites, such as an X-type or Y-type zeolite or zeolite beta.

In an embodiment, the zeolite materials suitable for this invention are characterized by silica to alumina ratio (Si/Al) of less than 1.5. In another embodiment, the zeolite materials are characterized by a Si/Al ratio ranging from 1.0 to 200, optionally from 1.0 to 100, optionally from 1.0 to 50, optionally from 1.0 to 10.

The present catalyst is adaptable to use in the various physical forms in which catalysts are commonly used. The catalyst of the invention may be used as a particulate material in a contact bed or as a coating material on structures having a high surface area. If desired, the catalyst can be deposited with various catalyst binder and/or support materials.

A catalyst comprising a substrate that supports a promoting metal or a combination of metals can be used to catalyze the reaction of hydrocarbons. The method of preparing the catalyst, pretreatment of the catalyst, and reaction conditions can influence the conversion, selectivity, and yield of the reactions.

The various elements that make up the catalyst can be derived from any suitable source, such as in their elemental form, or in compounds or coordination complexes of an organic or inorganic nature, such as carbonates, oxides, hydroxides, nitrates, acetates, chlorides, phosphates, sulfides and sulfonates. The elements and/or compounds can be prepared by any suitable method, known in the art, for the preparation of such materials.

The term “substrate” as used herein is not meant to indicate that this component is necessarily inactive, while the other metals and/or promoters are the active species. On the contrary, the substrate can be an active part of the catalyst. The term “substrate” would merely imply that the substrate makes up a significant quantity, generally 10% or more by weight, of the entire catalyst. The promoters individually can range from 0.01% to 60% by weight of the catalyst, optionally from 0.01% to 50%, optionally from 0.01% to 40%, optionally from 0.01% to 30%, optionally from 0.01% to 20%, optionally from 0.01% to 10%, optionally from 0.01% to 5%. If more than one promoter is combined, they together generally can range from 0.01% up to 70% by weight of the catalyst, optionally from 0.01% to 50%, optionally from 0.01% to 30%, optionally from 0.01% to 15%, optionally from 0.01% to 5%. The elements of the catalyst composition can be provided from any suitable source, such as in its elemental form, as a salt, as a coordination compound, etc.

The addition of a support material to improve the catalyst physical properties is possible. Binder material, extrusion aids or other additives can be added to the catalyst composition or the final catalyst composition can be added to a structured material that provides a support structure. For example, the final catalyst composition can include an alumina or aluminate framework as a support. Upon calcination these elements can be altered, such as through oxidation which would increase the relative content of oxygen within the final catalyst structure. The combination of the catalyst with additional elements such as a binder, extrusion aid, structured material, or other additives, and their respective calcination products, are included within the scope of the invention.

The catalyst can be prepared by combining a substrate with at least one promoter element. The substrate can be a molecular sieve, from either natural or synthetic sources. Zeolites and zeolite-like materials can be an effective substrate. Alternate molecular sieves also contemplated are zeolite-like materials such as the crystalline silicoaluminophosphates (SAPO) and the aluminophosphates (ALPO).

The method of catalyst preparation is not limited, and all suitable methods should be considered applicable. Particularly effective techniques are those utilized for the preparation of solid catalysts. Conventional methods include co-precipitation from an aqueous, an organic or a combination solution-dispersion, impregnation, dry mixing, wet mixing or the like, alone or in various combinations. In general, any method can be used which provides compositions of matter containing the prescribed components in effective amounts. According to an embodiment the substrate is charged with promoter via an incipient wetness impregnation. Other impregnation techniques such as by soaking, pore volume impregnation, or percolation can optionally be used. Alternate methods such as ion exchange, wash coat, precipitation, and gel formation can also be used. Various methods and procedures for catalyst preparation are listed in the technical report Manual of Methods and Procedures for Catalyst Characterization by J. Haber, J. H. Block and B. Dolmon, published in the International Union of Pure and Applied Chemistry, Volume 67, Nos 8/9, pp. 1257-1306, 1995, incorporated herein in its entirety.

The promoter elements can be added to or incorporated into the substrate in any appropriate form. In an embodiment, the promoter elements are added to the substrate by mechanical mixing, by impregnation in the form of solutions or suspensions in an appropriate liquid, or by ion exchange. In a more specific embodiment, the promoter elements are added to the substrate by impregnation in the form of solutions or suspensions in a liquid selected from the group of acetone, anhydrous (or dry) acetone, methanol, and aqueous solutions.

The promoter may be added to the substrate by ion exchange. Ion exchange may be performed by conventional ion exchange methods in which sodium, hydrogen, or other inorganic cations that may be typically present in a substrate are at least partially replaced via a fluid solution. In an embodiment, the fluid solution can include any medium that will solubilize the cation without adversely affecting the substrate. In an embodiment, the ion exchange may be performed by heating a solution containing any promoter selected from the group of Co, Mn, Ti, Zr, V, Nb, K, Cs, Ga, B, P, Rb, Ag, Na, Cu, Mg, Fe, Mo, Ce, and any combinations thereof in which the promoter(s) is(are) solubilized in the solution, which may be heated, and contacting the solution with the substrate. In another embodiment, the ion exchange includes heating a solution containing any one selected from the group of Ce, Cu, P, Cs, B, Co, or Ga, and any combinations thereof. In an embodiment, the solution may be heated to temperatures ranging from 50 to 120° C. In another embodiment, the solution is heated to temperatures ranging from 80 to 100° C.

The solution for use in the ion exchange method may include any fluid medium. A non-fluid ion exchange is also possible. In an embodiment, the solution for use in the ion exchange method includes an aqueous medium or an organic medium. In a more specific embodiment, the solution for use in the ion exchange method includes water.

The promoters may be incorporated into the substrate in any order or arrangement. In an embodiment, all of the promoters may be simultaneously incorporated into the substrate. In more specific embodiment, each promoter may be in an aqueous solution for ion-exchange with and/or impregnation to the substrate. In another embodiment, each promoter is in a separate aqueous solution, wherein each solution is simultaneously contacted with the substrate for ion-exchange with and/or impregnation to the substrate. In a further embodiment, each promoter is in a separate aqueous solution, wherein each solution is separately contacted with the substrate for ion-exchange with and/or impregnation to the substrate.

In an aspect, the at least one promoter includes boron. In an embodiment, the catalyst contains greater than 0.1 wt % boron based on the total weight of the catalyst. In another embodiment, the catalyst contains from 0.1 to 3 wt % boron, optionally from 0.1 to 1 wt % boron.

The boron promoter can be added to the catalyst by contacting the substrate, impregnation, or any other method, with any known boron source. In an embodiment, the boron source is selected from the group of boric acid, boron phosphate, methoxyboroxine, methylboroxine, and trimethoxyboroxine and combinations thereof. In another embodiment, the boron source may contain boroxines. In a further embodiment, the boron source is selected from the group of methoxyboroxine, methylboroxine, and trimethoxyboroxine and combinations thereof.

In an embodiment, a substrate may be previously treated with a boron source prior to an addition of at least one promoter, wherein the at least one promoter includes boron. In another embodiment, a boron treated zeolite may be combined with at least one promoter, wherein the at least one promoter includes boron. In a further embodiment, boron may be added to the catalyst system by adding at least one promoter containing boron as a co-feed with toluene and methanol. In an even further embodiment, boron may be added to the catalyst system by adding boroxines as a co-feed with toluene and methanol. The boroxines can include, methoxyboroxine, methylboroxine, and trimethoxyboroxine, and combinations thereof. The boron treated zeolite further combined with at least one promoter including boron may be used in preparing a supported catalyst such as extrudates and tablets.

When slurries, precipitates or the like are prepared, they may be dried, usually at a temperature sufficient to volatilize the water or other carrier, such as from 100° C. to 250° C., with or without vacuum. Irrespective of how the components are combined and irrespective of the source of the components, the dried composition is generally calcined in the presence of an oxygen-containing gas, usually at temperatures between about 300° C. and about 900° C. for from 1 to 24 hours. The calcination can be in an oxygen-containing atmosphere, or alternately in a reducing or inert atmosphere.

The prepared catalyst can be ground, pressed, sieved, shaped and/or otherwise processed into a form suitable for loading into a reactor. The reactor can be any type known in the art, such as a fixed bed, fluidized bed, or swing bed reactor. Optionally an inert material can be used to support the catalyst bed and to place the catalyst within the bed. Depending on the catalyst, a pretreatment of the catalyst may, or may not, be necessary. For the pretreatment, the reactor can be heated to elevated temperatures, such as 200° C. to 900° C. with an air flow, such as 100 mL/min, and held at these conditions for a length of time, such as 1 to 3 hours. Then, the reactor can be brought to the operating temperature of the reactor, for example 300° C. to 550° C., or optionally down to any desired temperature, for instance down to ambient temperature to remain under a purge until it is ready to be put in service. The reactor can be kept under an inert purge, such as under a nitrogen or helium purge.

Embodiments of reactors that can be used with the present invention can include, by non-limiting examples: fixed bed reactors; fluid bed reactors; and entrained bed reactors. Reactors capable of the elevated temperature as described herein, and capable of enabling contact of the reactants with the catalyst, can be considered within the scope of the present invention. Embodiments of the particular reactor system may be determined based on the particular design conditions and throughput, as by one of ordinary skill in the art, and are not meant to be limiting on the scope of the present invention. An example of a suitable reactor can be a fluid bed reactor having catalyst regeneration capabilities. This type of reactor system employing a riser can be modified as needed, for example by insulating or heating the riser if thermal input is needed, or by jacketing the riser with cooling water if thermal dissipation is required. These designs can also be used to replace catalyst while the process is in operation, by withdrawing catalyst from the regeneration vessel from an exit line or adding new catalyst into the system while in operation.

In another aspect, the one or more reactors may include one or more catalyst beds. In the event of multiple beds, an inert material layer can separate each bed. The inert material can comprise any type of inert substance. In an embodiment, a reactor includes between 1 and 25 catalyst beds. In a further embodiment, a reactor includes between 2 and 10 catalyst beds. In a further embodiment, a reactor includes between 2 and 5 catalyst beds. In addition, the co-feed, the C₁ source and/or toluene may be injected into a catalyst bed, an inert material layer, or both. In a further embodiment, at least a portion of the C₁ source and at least a portion of the co-feed are injected into a catalyst bed(s) and at least a portion of the toluene feed is injected into an inert material layer(s).

In an alternate embodiment, the entire C₁ source is injected into a catalyst bed(s), all of the toluene feed is injected into an inert material layer(s) and all of the co-feed is injected into one of: the catalyst bed(s), the inert material layer(s), or any combination thereof. In another aspect, at least a portion of the toluene feed is injected into a catalyst bed(s), at least a portion of the co-feed is injected into a catalyst bed(s), and at least a portion the C₁ source is injected into an inert material layer(s).

The toluene and C₁ source coupling reaction may have a toluene conversion percent greater than 0.01 mol %. In an embodiment the toluene and C₁ source coupling reaction is capable of having a toluene conversion percent in the range of from 0.05 mol % to 40 mol %. In a further embodiment the toluene and C₁ source coupling reaction is capable of having a toluene conversion in the range of from 2 mol % to 40 mol %, optionally from 5 mol % to 35 mol %, optionally from 10 mol % to 30 mol %.

In an aspect the toluene and C₁ source coupling reaction is capable of selectivity to styrene greater than 1 mol %. In another aspect, the toluene and C₁ source coupling reaction is capable of selectivity to styrene in the range of from 1 mol % to 99 mol %. In an aspect the toluene to a C₁ source coupling reaction is capable of selectivity to ethylbenzene greater than 1 mol %. In another aspect, the toluene and C₁ source coupling reaction is capable of selectivity to ethylbenzene in the range of from 1 mol % to 99 mol %. In an aspect the toluene and C₁ source coupling reaction is capable of yielding less than 0.5 mol % of ring alkylated products such as xylenes.

While illustrative embodiments have been depicted and described, modifications thereof can be made by one skilled in the art without departing from the spirit and scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

The term “conversion” refers to the percentage of reactant (e.g. toluene) that undergoes a chemical reaction.

X_{Tol}=conversion of toluene (mol %)=(Tolᵢₙ−Tolₒᵤₜ)/Tolᵢₙ×100

X_{MeOH}=conversion of methanol to styrene+ethylbenzene (mol %)=(MeOHᵢₙ−MeOHₒᵤₜ)/MeOHᵢₙ×100

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

The term “selectivity” refers to the relative activity of a catalyst in reference to a particular compound in a mixture. Selectivity is quantified as the proportion of a particular product relative to all other products.

S_{Sty}=selectivity of toluene to styrene (mol %)=Styₒᵤₜ/Tol_{converted}×100

S_{Bz}=selectivity of toluene to benzene (mol %)=Benzeneₒᵤₜ/Tol_{converted}×100

S_{EB}=selectivity of toluene to ethylbenzene (mol %)=EBₒᵤₜ/Tol_{converted}×100

S_{Xyl}=selectivity of toluene to xylenes (mol %)=Xylenesₒᵤₜ/Tol_{converted}×100

S_{Sty+EB} (MEOH)=selectivity of methanol to styrene+ethylbenzene (mol %)=(Styₒᵤₜ+EBₒᵤₜ)/MeOH_{converted}×100

The term “zeolite” refers to a molecular sieve containing an aluminosilicate lattice, usually in association with some aluminum, boron, gallium, iron, and/or titanium, for example. In the following discussion and throughout this disclosure, the terms molecular sieve and zeolite will be used more or less interchangeably. One skilled in the art will recognize that the teachings relating to zeolites are also applicable to the more general class of materials called molecular sieves.

The various aspects of the present invention can be joined in combination with other aspects of the invention and the listed embodiments herein are not meant to limit the invention. All combinations of various aspects of the invention are enabled, even if not given in a particular example herein.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the aspects and embodiments disclosed herein are usable and combinable with every other embodiment and/or aspect disclosed herein, and consequently, this disclosure is enabling for any and all combinations of the embodiments and/or aspects disclosed herein. Other and further embodiments, versions and examples of the invention may be devised without departing from the basic scope thereof and the scope thereof is determined by the claims that follow.

What is claimed is:

1. A process for making styrene in a pre-existing facility comprising an infrastructure capable of producing styrene, wherein the infrastructure comprises at least one dehydrogenation unit, the process comprising:
providing an alkylation unit comprising an alkylation reactor coupled to the infrastructure;
contacting toluene with a C₁ source in the presence of a first catalyst and a co-feed in the alkylation reactor to form a first product stream comprising styrene and ethylbenzene;
wherein the styrene and ethylbenzene from the first product stream are routed for further processing to a portion of the pre-existing facility.

2. The process of claim 1, further comprising forming the co-feed from the separation of a mixture of hydrogen and carbon monoxide, the mixture being separated from an initial product stream comprising styrene and ethylbenzene, the initial product stream formed from toluene contacting the C₁ source in the presence of an initial catalyst in the alkylation reactor.

3. The process of claim 1, wherein the infrastructure further comprises a plurality of infrastructure reactors connected in parallel with the alkylation reactor.

4. The process of claim 3, wherein the infrastructure reactors are configured to process at least an ethylene feedstock.

5. The process of claim 2, wherein the co-feed comprises carbon monoxide.

6. The process of claim 5, wherein the alkylation unit further comprises a ceramic membrane configured to separate the mixture of hydrogen and carbon monoxide.

7. The process of claim 1, wherein the C₁ source is selected from the group consisting of methanol, formaldehyde, formalin, trioxane, methylformcel, paraformaldehyde, methylal, dimethyl ether, and combinations thereof.

8. The process of claim 1, wherein the first catalyst comprises at least one promoter on a support material.

9. The process of claim 8, wherein the at least one promoter is selected from the group consisting of Co, Mn, Ti, Zr, V, Nb, K, Cs, Ga, B, P, Rb, Ag, Na, Cu, Mg, Fe, Mo, Ce, and combinations thereof.

10. The process of claim 8, wherein the at least one promoter is selected from the group consisting of Ce, Cu, P, Cs, B, Co, Ga, and combinations thereof.

11. The process of claim 8, wherein the support material comprises a zeolite.

12. The process of claim 1, wherein the first catalyst comprises B and Cs supported on a zeolite.

13. A process for making ethylbenzene and/or styrene comprising:
reacting toluene and a C₁ source in the presence of a co-feed in one or more reactors to form a first product stream comprising one or more of ethylbenzene, styrene, toluene, methanol, hydrogen, and carbon monoxide;
separating the ethylbenzene from the first product stream; and
sending the ethylbenzene to a portion of a pre-existing facility; and
forming styrene from the ethylbenzene in the pre-existing facility.

14. The process of claim 13, further comprising:
separating a mixture of the hydrogen and carbon monoxide from the first product stream;
separating at least a portion of the carbon monoxide from the mixture and recycling the carbon monoxide as the co-feed;
sending ethylbenzene to a dehydrogenation unit in the pre-existing facility, wherein styrene is formed from the dehydrogenation of ethylbenzene.

15. The process of claim 14, further comprising:
separating styrene from the first product stream; and
sending the styrene and ethylbenzene to a separation unit in the pre-existing facility.

16. A method of revamping an existing styrene production facility comprising:
coupling one or more reactors to an existing styrene production facility, wherein the reactor is capable of reacting toluene with methanol in the presence of a co-feed to produce a first product stream comprising ethylbenzene and/or styrene.

17. The method of claim 16, further comprising sending the first product stream comprising ethylbenzene and/or styrene to the existing styrene production facility for further processing to form styrene.

18. The method of claim 17, wherein the existing styrene production facility comprises a separation apparatus to remove at least a portion of any benzene from the first product stream, an alkylation reactor to form ethylbenzene by reacting benzene and ethylene, and a dehydrogenation reactor to form styrene by dehydrogenating ethylbenzene.

19. The method of claim 16, wherein the first product stream comprises one or more of benzene, toluene or methanol and at least a portion of the methanol is separated from the first product stream and recycled to the one or more reactors.

20. The method of claim 16, wherein the first product stream comprises one or more of hydrogen, carbon monoxide, toluene or methanol and at least a portion of the carbon monoxide is separated from the first product stream and recycled to the one or more reactors.
